(19) United States
(12) Patent Application Publication
(10) Pub. No.: US 2017/0181743 A1
(43) Pub. Date: Jun. 29, 2017

(54) THREAD IMPLANTATION ROD FOR PREVENTING DAMAGE TO SUBCUTANEOUS TISSUE AND BLOOD VESSELS

(71) Applicant: Gyung Gyun KIM, Seoul (KR)

(72) Inventors: Gyung Gyun KIM, Seoul (KR); Han Jin KWON, Seoul (KR)

(21) Appl. No.: 15/304,870

(22) PCT Filed: Apr. 17, 2015
(86) PCT No.: PCT/KR2015/003870

§ 371 Date: Oct. 18, 2016

(30) Foreign Application Priority Data
Apr. 18, 2014 (KR) 10-2014-0046807

(51) Int. Cl. A61B 17/04; A61B 17/06; A61B 17/34

(52) U.S. Cl. CPC A61B 17/0469; A61B 17/34; A61B 17/06066; A61B 2017/061

(57) ABSTRACT

The present invention relates to a thread implantation rod and relates to a needle for thread-embedding therapy having a tubular shape for allowing a suture to be inserted and moved therein, wherein the end is rounded and blocked and also has a sharp shape so as to prevent the skin, muscle and blood vessels from being torn during an operation, thereby preventing damage to subcutaneous tissue.

### TECHNICAL FIELD

The present invention relates to a thread implantation rod, and in particular to a thread implantation rod for preventing a damage to a subcutaneous tissue which has a tubular shape for allowing a suture to be inserted and moved therein, wherein the end is rounded and blocked and also has a sharp shape so as to prevent the skin, muscle and blood vessels from being torn during an operation, thereby preventing damage to the subcutaneous tissue.

### BACKGROUND ART

In recent years, an operation, for example, a thread therapy, is increasingly used in the field of a dermatology and a plastic surgery, wherein a thread a part of which has been inserted in an injection needle is inserted under a subcutaneous tissue for the sake of a winkle prevention, a muscle recovery and the recovery of a skin tissue, and only the inserted injection needle is removed, while leaving behind the thread in the subcutaneous tissue.

According to the contents of a thread insertion apparatus for a wrinkle prevention plastic surgery (Korean patent registration number 10-1261175, a patent document 1) which corresponds to a technology related with the injection needle used in the aforementioned method, the apparatus is formed in an empty tubular shape through which a suture can passes, and since it is inserted passing through a skin, the front end thereof is made sharp.

Since the front end thereof is sharp, a muscle, a tissue or a blood vessel may be damaged and injured during an insertion procedure of an injection needle.

According to the contents of an injection needle type sewing apparatus (Korean patent registration number 10-0166449, a patent document 2) which corresponds to another technology related with an injection needle allowing a thread to be inserted into a subcutaneous tissue together with an injection needle, a hole is perforated at one side of an outer surface of an injection needle or a shoulder is formed through a cutting procedure, by which an end of an intermediate portion of the needle is hooked and can be easily inserted.

In the aforementioned patent document 2 configuration, the thread can be smoothly inserted into the subcutaneous tissue together with the injection needle.

In the aforementioned patent document 2 configuration, since the end thereof is made sharp, a subcutaneous tissue or a blood vessel may be injured during an insertion procedure of the injection needle.

In particular, according to the configurations of the patent documents 1 and 2, when an injection needle is taken out after it has been inserted in a subcutaneous tissue, the inner side and ends are made open so as to prevent the thread from being taken out together with the injection needle. In this case, a subcutaneous tissue or a blood vessel may be injured by a sharp needle end, and the damaged subcutaneous tissue or blood vessel may be inserted into the inside of the tube through the opened portion, thus accelerating the damages, for which the dead tissue or blood vessel may gather near the suture which has been in the inside of the tube, and the dead tissue, etc. may position near the suture, so the color of the skin may be changed and protruded, not even. A cyst may be formed due to the dead tissue, thus causing a problem.

### PRIOR ART DOCUMENTS

1. Patent document 1: KR 10-1261175 (Apr. 29, 2013)

2. Patent document 2: KR 10-0166449 (Sep. 23, 1998)

### DISCLOSURE OF INVENTION

Accordingly, it is an object of the present invention to provide a thread implantation rod for preventing damage to a subcutaneous tissue and blood vessels which is able to resolve the problems encountered in a conventional technology, wherein different from a conventional technology wherein an end is open, the present invention adapts a closed end structure, thus preventing any damage to a subcutaneous tissue or a blood vessel or any inflow thereof into the inside of a body, and an end is tapered sharp, and an apex is rounded, by which it can squeeze into a subcutaneous tissue, not like a conventional sharp end tearing off a subcutaneous tissue or a blood vessel. In this way, it is possible to prevent to the maximum any damage of a subcutaneous tissue and a blood vessel during a subcutaneous tissue insertion procedure of an implantation rod.

Moreover, the structure wherein an end is closed when an implantation rod is taken out after it has been inserted into a subcutaneous tissue, may contribute to preventing an inserted suture from being taken out together with an implantation rod since the suture is hooked.

More specifically, one side of an outer surface is cut away, thus forming a cut-away part, and the cut-away part may form a one-side inclined part and an other-side inclined part which are inclined downward toward the center in the direction where they are being opposite to each other, and the position of the cut-away part is set at a boundary portion between the tapered part and the body part, and the top of the other-side inclined part at the side of the tapered part positions lower than the top of the one-side inclined part which positions at the side of the body part. In this way, in a state where the implantation rod wherein a part of the suture has been inserted, is inserted in a subcutaneous tissue, if the implantation rod is moved in the direction opposite to when the entering direction while pressing a little the side of the body from the outside, the folded portion of the suture may be lifted upward along the other-side inclined part, so the end of the suture cannot be taken out together with the implantation rod without any interference with the tapered part the end of which is closed.

In addition, since the guide protrusions protruding from the inner surface of the rear side of the cut-away part are formed, it is possible to guide the folded portion of the suture when it is lifted upward when taking out the implantation rod from the subcutaneous tissue.

Since an assistant part is formed at a portion opposite to the rounded part, it is possible to prevent the folded portion of the suture from positioning at the inner side of the tapered part when taking out the implantation rod, which makes it possible to more surely prevent the suture from coming out together.

Furthermore, the tapered part is formed in the type of a metallic stick the inside of which is filled, thus forming an inclined surface surrounded by the other-side inclined part, by which the folded portion of the suture which is supposed to be lifted upward, can be smoothly and easily lifted upward with the aid of the pressing, which makes it possible to easily take out only the implantation rod from the subcutaneous tissue.

To achieve the above objects, there is provided a thread implantation rod for preventing damage to a subcutaneous tissue and blood vessels, which may include, but is not limited to, a body part 10 which is formed of an empty cylindrical tubular body made of a metallic material, wherein an opening part 11 is formed at an end of one side thereof; a tapered part 20 which is formed integral with the body part 10 at an end of the other side of the body part 10 and is formed in a conical shape wherein the diameter thereof is gradually decreased in the direction of an end thereof, and a rounded part 21 is formed at the end thereof and is closed in a convex curve shape; and a cut-away part 30 which is formed cut-away from one side of the body part 10 to one side of the tapered part 20 and includes a one-side inclined part 31 which is formed inclined downward at a predetermined angle from an outer surface of the body part 10, wherein a lower end thereof positions higher than a central axis (a) of the body part 10; a horizontal part 32 which is connected to a front end of the one-side inclined part 31 and is formed in the direction of the tapered part 20 in parallel with the central axis (a) of the body part 10; and an other-side inclined part 33 which is formed inclined upward at a predetermined angle from a front end of the horizontal part 32, wherein the top thereof extends to an outer surface of the tapered part 20, and the top thereof positions lower than the top of the one-side inclined part 31.

Herein, the body part 10 is equipped with a guide protrusion 40 formed protruding in the direction of the center at an inner surface of the rear side of the cut-away part 30.

Moreover, the opposite side of the rounded part 21 is equipped with an assistant rounded part 22 having a large diameter curvature than the rounded part 21, wherein the assistant part 22 is formed in a convex curve shape.

In addition, the tapered part 20 is formed in a metallic stick type the inside of which is filled, and is equipped with an inclined surface 23 surrounded by the other-side inclined part 33.

### Advantageous Effects of the Invention

According to the present invention, different from a conventional technology wherein an end is open, the present invention adapts a closed end structure, thus preventing any damage to a subcutaneous tissue or a blood vessel or any inflow thereof into the inside of a body, and an end is tapered sharp, and an apex is rounded, by which it can squeeze into a subcutaneous tissue, not like a conventional sharp end tearing off a subcutaneous tissue or a blood vessel. In this way, it is possible to prevent to the maximum any damage of a subcutaneous tissue and a blood vessel during a subcutaneous tissue insertion procedure of an implantation rod.

The structure wherein an end is closed when an implantation rod is taken out after it has been inserted into a subcutaneous tissue, will contribute to preventing an inserted suture from being taken out together with an implantation rod since the suture is hooked.

More specifically, one side of an outer surface is cut away, thus forming a cut-away part, and the cut-away part may form a one-side inclined part and an other-side inclined part which are inclined downward toward the center in the direction where they are being opposite to each other, and the position of the cut-away part is set at a boundary portion between the tapered part and the body part, and the top of the other-side inclined part at the side of the tapered part positions lower than the top of the one-side inclined part which positions at the side of the body part. In this way, in a state where the implantation rod wherein a part of the suture has been inserted, is inserted in a subcutaneous tissue, if the implantation rod is moved in the direction opposite to when the entering direction while pressing a little the side of the body from the outside, the folded portion of the suture may be lifted upward along the other-side inclined part, so the end of the suture cannot be taken out together with the implantation rod without any interference with the tapered part the end of which is closed.

In addition, since the guide protrusions protruding from the inner surface of the rear side of the cut-away part are formed, it is possible to guide the folded portion of the suture when it is lifted upward when taking out the implantation rod from the subcutaneous tissue.

Furthermore, since an assistant part is formed at a portion opposite to the rounded part, it is possible to prevent the folded portion of the suture from positioning at the inner side of the tapered part when taking out the implantation rod, which makes it possible to more surely prevent the suture from coming out together.

In particular, the tapered part is formed in the type of a metallic stick the inside of which is filled, thus forming an inclined surface surrounded by the other-side inclined part, by which the folded portion of the suture which is supposed to be lifted upward, can be smoothly and easily lifted upward with the aid of the pressing, which makes it possible to easily take out only the implantation rod from the subcutaneous tissue.

### BRIEF DESCRIPTION OF DRAWINGS

FIG. 1 is a side cross sectional view illustrating a thread implantation rod for preventing a damage to a subcutaneous tissue and blood vessels according to the present invention.

FIG. 2 is a perspective view illustrating a thread implantation rod for preventing damage to a subcutaneous tissue and blood vessels according to the present invention.

FIGS. 3 to 5 are schematic cross sectional views illustrating a commercial use state of a thread implantation rod for preventing damage to a subcutaneous tissue and blood vessels according to the present invention.

FIG. 6 is a cross sectional view illustrating another example of a thread implantation rod for preventing damage to a subcutaneous tissue and blood vessels according to the present invention.

FIG. 7 is a disassembled perspective view illustrating a state where an inclined surface is formed according to the embodiment in FIG. 6.

### LEGEND OF REFERENCE NUMBERS

1: Finger

2: Skin

3: Suture

10: Body part

11: Opening part

12: Handle

20: Tapered part

21: Rounded part

22: Assistant rounded part

23: Inclined surface

30: cut-away part

31: One-side inclined part

32: Horizontal part

33: Other-side inclined part

40: Guide protrusion

“a”: Central shaft

### MODES FOR CARRYING OUT THE INVENTION

The thread implantation rod for preventing damage to a subcutaneous tissue and blood vessels according to the present invention will be described with reference to the accompanying drawings.

The thread implantation rod for preventing damage to a subcutaneous tissue and blood vessels according to the present invention may include, but is not limited to a body part 10, a tapered part 20 and a cut-away part 30.

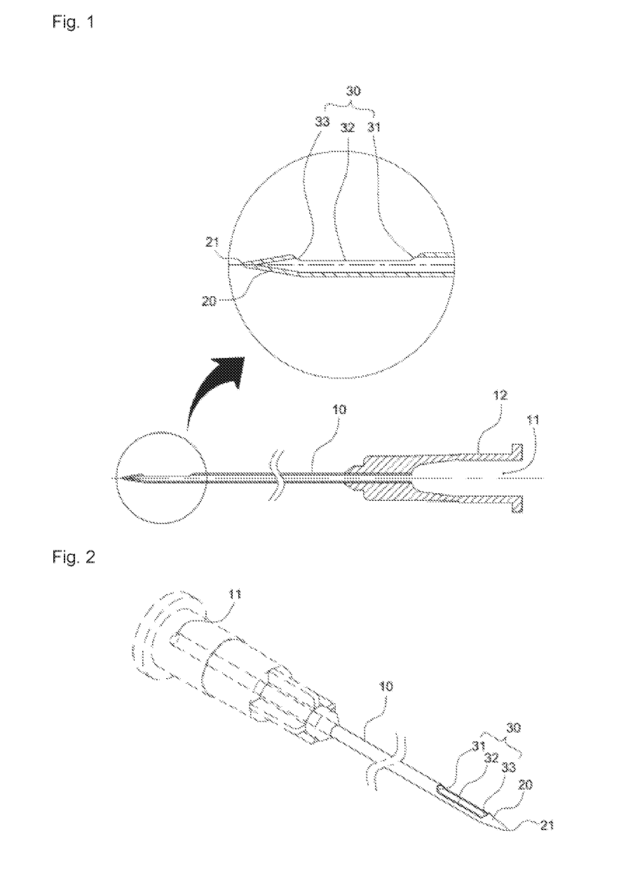

Referring to FIGS. 1 and 2, the body part 10 is formed of an empty cylindrical tubular body of a metallic material, wherein an opening part 11 is formed at an end of one side thereof.

It is preferred that a handle 12 may be installed at a portion where the opening part 11 is formed as illustrated in FIG. 1.

A suture 3 may be inserted through the opening part 11.

The suture 3 may be made of a variety of threads, for example, a biodegradable material, an undegraded material, etc.

An intermediate portion of the suture 3 is folded like in a typical thread therapy method. One strand divided from the folded portion may be inserted through the opening part 11.

Moreover, the body part 10 may be equipped with a guide protrusion 40 protruding toward the center from the inner surface of the rear side of the cut-away part 30.

The guide protrusion 40 is configured in such a way that if an implantation rod is inserted into the skin 2 with a suture 3 being inserted therein, and the implantation rod is taken out, the lower side of the suture 3 inserted in the body part 10 is hooked by the guide protrusion 40 and is lifted up. At this time, the guide protrusion 40 may guide the folded portion of the suture 3 to be taken out toward the outside of the cut-away part 30, not positioning inside the tapered part 20.

As illustrated in FIGS. 1 and 2, the tapered part 20 is formed integral with the body part 10 at the end of the other side of the body part 10.

This tapered part 20 is formed in a conical shape wherein the diameter is gradually decreased in the direction opposite to the body part 10.

The conical tapered part 20 is formed in a typical needle or pin shape which allows for an easier insertion of the implantation rod into the skin.

A rounded part 21 formed in a blocked convex and curve shape may be formed at an end of the taper part 20.

The formation of the rounded part 21 may allow the tapered part 20 to squeeze into the subcutaneous tissue, by which the subcutaneous tissue or blood vessel can be prevented from tearing off.

The opposite side of the rounded part 21 may be equipped with an assistant rounded part 22 which has a larger curvature diameter than the rounded part 21 and is formed in a convex curve shape.

The formation of the assistant rounded part 22 is intended to prevent the folded portion of the suture 3 from being hooked by the implantation rod and coming out together with the implantation rod since the folded portion of the suture 3 is inserted into the inner side of the tapered part 20 when the implantation rod moves in the direction opposite to the insertion direction into the skin if the tapered part 20 is made of an empty tubular body or a space is formed inside of the tapered part 20.

As illustrated in FIGS. 1 and 2, the cut-away part 30 is formed cut-away from one side of the body part 10 to one side of the tapered part 20.

This cut-away part 30 may be formed of a one-side inclined part 31, a horizontal part 32, and another-side inclined part 33 which are referred to the shapes of the side cross sections thereof.

The boundaries between the one-side inclined part 31, the horizontal part 32 and the other-side inclined part 33 may be processed with a smooth curve line.

As illustrated in the drawing, the one-side inclined part 31 may be formed inclined downward at a predetermined angle from the outer surface of the body part 10, wherein the lower side thereof positions higher than the central axis (a) of the body part 10.

The horizontal part 32 may be connected to a front end of the one-side inclined part 31 and may be formed in the direction of the tapered part 20 in parallel with the central axis (a) of the body part 10.

The other-side inclined part 33 may be formed inclined upward at a predetermined angle from the front end of the horizontal part 32, wherein the upper side thereof extends to the outer surface of the tapered part 20.

The upper side of the other-side inclined part 33 may be formed lower than the upper side of the one-side part 31.

In the cut-away part 30, one side of the suture 3 may position inside of the body part 10 with respect to the folded portion of the suture 3, and the other side thereof may position separated outward of the body part 10.

In the present invention, the shape of the cut-away part 30 is limited to the aforementioned configuration, by which when the implantation rod is taken out, a part of the suture 3 positioned inside of the body part 10 can be easily taken out to the outside of the body part 10 through the cut-away part 30.

Since the position of the other-side inclined part 33 is tapered, it can position at the tapered part 20 having the smaller diameter than the body part 10, so the folded portion of the suture 3 can be easily taken out to the outside of the cut-away part 30.

When viewing from the side surface, the horizontal part 32 may position higher than the central axis (a) of the body part 10, so the length that the cut-away part 30 occupies at the circumference of the body part 10 can be reduced, by which a phenomenon that the cut-away part 30 is interfered with or is hooked by a subcutaneous tissue or a blood vessel can be prevented.

Moreover, the one-side inclined part 31 may be formed inclined in the direction opposite to the other-side inclined part 33, so a part of the suture 3 protruding outward from the cut-away part 30 can be prevented from tearing off or being hooked.

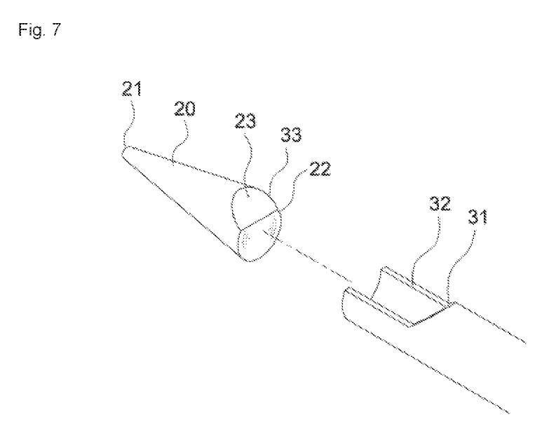

As illustrated in FIGS. 6 and 7, the tapered part 20 is made of a metallic stick type the inside of which is filled, and as illustrated in FIG. 7, it may be configured in such a way that an inclined surface 23 surrounded by the other-side inclined part 33 can be formed.

This configuration is intended to allow the suture 3, which is discharged along the other-side inclined part 33, to be discharged smoothly as if it slides.

A plurality of barbs, in general, are formed on the surface of the suture 3 for a thread therapy. If the tapered part 20 is made of a tubular body, and a shoulder is accordingly formed with the thickness corresponding to the tubular body at the other-side inclined part 33, the barbs may be hooked by the shoulder, which may cause an interference when the suture 3 is smoothly discharged.

This configuration may contribute to preventing the formation of such a shoulder and may allow the other-side inclined part 33 to be equipped with a smooth surface, by which the bars are not hooked by the shoulder, and the suture 3 can be smoothly discharged.

In the configuration in FIG. 7, the body part 10 and the tapered part 20 may be engaged integral.

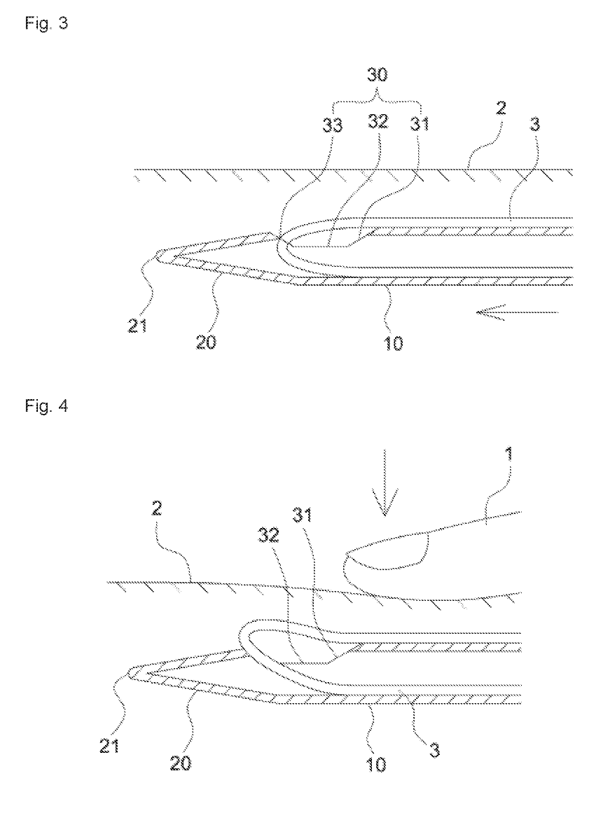

The operation of the thread implantation rod for preventing a subcutaneous tissue and blood vessels according to the present invention will be described with reference to FIGS. 3 to 5.

FIG. 3 is a view illustrating a state where a thread implantation rod is inserted inside of the skin 2 in a state where one end of the suture 3 is folded and inserted inside of the body part 10 of the thread implantation rod of the present invention.

A part of the folded portion of the suture 3 may position inside of the body part 10 through the cut-away part 30, and another part thereof may position at the subcutaneous tissue outside the body part 10.

FIG. 4 is a view illustrating a state where the skin surface is pressed with a finger 1 so as to take out the thread implantation rod.

If the skin surface is pressed with a finger, namely, it is pressed to correspond to a portion where the rear side of the cut-away part 30 positions, the suture 3 protruding outward of the body part 10 may be pressed by a skin elasticity, and the folded portion may be lifted up, so all or a part of the folded portions may protrude outward of the body part 10 through the cut-away part 30.

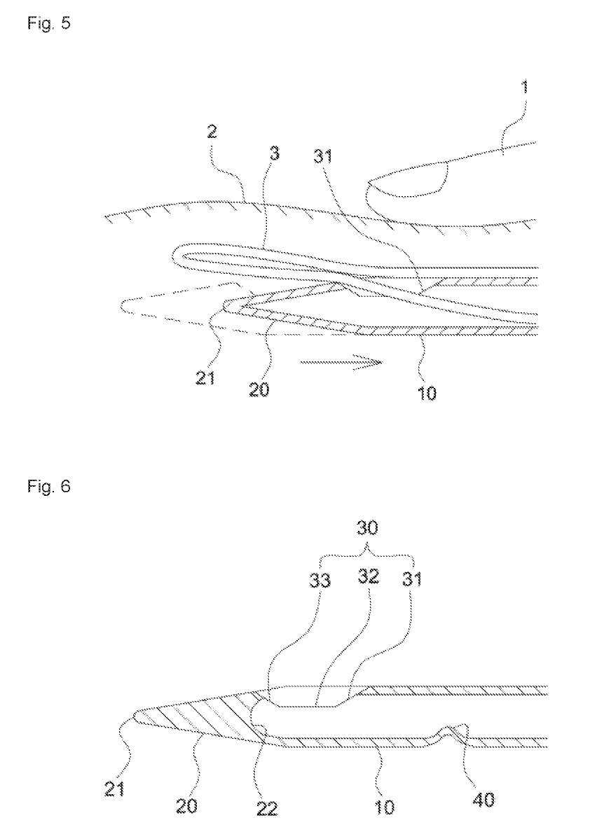

In this state, as illustrated in FIG. 5, if the implantation rod is taken out, the portion of the suture 3 positioned inside of the body part 10 can be supported and guided by the other-side guide part 33, and consequently, only the implantation rod will be taken out, and the portion of the suture 3 inside the body part 10 may position inside of the subcutaneous tissue.

The formations of the guide protrusion 40, the assistant rounded part 22 and the inclined surface 23 may further allow only the implantation rod to be taken out from the inside.

### INDUSTRIAL APPLICABILITY

In the present invention, an injection can be carried out in a state where a part of a thread is inserted in an injection needle, and only an injection needle can be taken out while leaving behind the thread in the subcutaneous tissue, so the present invention may be used for a dermatology, a plastic surgery, etc. which allows for a wrinkle prevention, a muscle recovery, a skin tissue recovery, etc.

1. A thread implantation rod for preventing damage to a subcutaneous tissue and blood vessels, comprising:
a body part (10) which is formed of an empty cylindrical tubular body made of a metallic material, wherein an opening part (11) is formed at an end of one side thereof;
a tapered part (20) which is formed integral with the body part (10) at an end of the other side of the body part (10) and is formed in a conical shape wherein the diameter thereof is gradually decreased in the direction of an end thereof, and a rounded part (21) is formed at the end thereof and is closed in a convex curve shape; and
a cut-away part (30) which is formed cut-away from one side of the body part 10 to one side of the tapered part (20) and includes:
a one-side inclined part (31) which is formed inclined downward at a predetermined angle from an outer surface of the body part (10), wherein a lower end thereof positions higher than a central axis (a) of the body part (10);
a horizontal part (32) which is connected to a front end of the one-side inclined part (31) and is formed in the direction of the tapered part (20) in parallel with the central axis (a) of the body part (10); and
an other-side inclined part (33) which is formed inclined upward at a predetermined angle from a front end of the horizontal part (32), wherein the top thereof extends to an outer surface of the tapered part (20), and the top thereof positions lower than the top of the one-side inclined part (31).

2. The rod of claim 1, wherein the body part (10) is equipped with a guide protrusion (40) formed protruding in the direction of the center at an inner surface of the rear side of the cut-away part (30).

3. The rod of claim 2, wherein the opposite side of the rounded part (21) is equipped with an assistant rounded part (22) having a large diameter curvature than the rounded part (21), wherein the assistant part (22) is formed in a convex curve shape.

4. The rod of claim 1, wherein the tapered part (20) is formed in a metallic stick type the inside of which is filled, and is equipped with an inclined surface (23) surrounded by the other-side inclined part (33).

5. The rod of claim 2, wherein the tapered part (20) is formed in a metallic stick type the inside of which is filled, and is equipped with an inclined surface (23) surrounded by the other-side inclined part (33).

6. The rod of claim 3, wherein the tapered part (20) is formed in a metallic stick type the inside of which is filled, and is equipped with an inclined surface (23) surrounded by the other-side inclined part (33).
